(19) United States
(12) Patent Application Publication
(10) Pub. No.: US 2025/0132054 A1
(43) Pub. Date: Apr. 24, 2025

(54) System and Method for Determination of Cause of Death from Clinical Information

(71) Applicant: Veritas Data Research, Inc., Claymont, DE (US)

(72) Inventors: Shahir Kassam-Adams, Lovingston, VA (US); Kirstian Macaulay, Edinburgh (GB); Jason LaBonte, W. Lafayette, IN (US); Omnya El Massad, North York (ON)

(21) Appl. No.: 18/918,213

(22) Filed: Oct. 17, 2024

Related U.S. Application Data
(60) Provisional application No. 63/545,272, filed on Oct. 23, 2023.

(51) Int. Cl. G16H 50/70; G16H 10/60

(52) U.S. Cl. CPC G16H 50/70; G16H 10/60

(57) ABSTRACT

Systems and methodologies for determining the likely cause of death of an individual from the individual's clinical history are disclosed. In particular, embodiments of the present disclosure relate to the processing of medical information through one or more algorithms, which can assess clinical and non-clinical variables to determine the probability that one or more medical or clinical factors in an individual's history is the precipitating cause of death, along with optionally identifying secondary potential causes of death and the assignment of a confidence variable, score and/or level indicating the calculated reliability of the assignment. The output data may be used, for example, in or with systems and methods with improved actuarial risk predictions and more accurate drug effectiveness and survival curves, among other applications.

### CROSS-REFERENCE TO RELATED APPLICATIONS

The present application claims priority to U.S. Provisional Patent Application No. 63/545,272, filed on Oct. 23, 2023, the disclosure of which is hereby incorporated by reference herein in its entirety.

### TECHNICAL FIELD

The present disclosure relates generally to modeled data about individuals, and specifically to computer-implemented systems and methods deriving or inferring cause of death about specific individuals.

### BACKGROUND AND SUMMARY

Death is a critical endpoint in health research and related research disciplines, and further provides a vital statistic for commercial operations such as, for example, triggering payments for life insurance benefits. However, mortality data is poorly characterized and limited in availability. While the fact of death, i.e., whether an individual is deceased and when that event occurred, is reasonably accurate and can be assembled from public sources such as obituaries, the cause of death is rarely available in public notices. More importantly, even the cause of death listed in official death certificates is often inaccurate. Studies have found that over one-third of death certificates list an incorrect underlying cause of death.

Cause of death is often determined by a coroner or medical examiner; however, the required qualifications for this individual can vary from an elected coroner as young as eighteen years old or a highly trained physician appointed as medical examiner. Currently, the only sources of these causes of death are state-level registries of death certificates and the Center for Disease Control and Prevention's (CDC) aggregated repository of those same certificates, which is not available for commercial use, among other disadvantages. A widely-accessible cause of death dataset and associated computer-implemented systems and methods are needed for academic and commercial organizations to perform important research and accurately service customers.

There is a need to understand the cause of death for numerous applications, including health research, risk modeling for insurance and benefits, and public health surveillance. In particular, there is a need to understand the likely cause of death at the individual level to allow correlation to a deceased individual's specific environmental, demographic, socioeconomic, behavioral, and health attributes. Rather than relying on the inaccurate and inaccessible cause of death information listed in death certificates, embodiments of the present disclosure employ different data source(s) alongside an advanced processing workflow to assess a cause of death data set at the individual level.

There is further a need for improvements for enabling healthcare data sets within healthcare records of deceased individuals to be accessible and usable for accurately determining cause of death. Embodiments of the present disclosure provide an advancement made in computer technology consisting of improvements defined by logical structures and processes directed to a specific implementation of a solution to a problem in software, data structures and data management, wherein the existing data structure technology relies upon unacceptable and inaccurate cause of death determinations. In particular, embodiments of the present disclosure provide a system and method that creates a specific, non-abstract improvement to computer functionality employing deidentified data to assess cause of death for individuals.

In various embodiments, the present disclosure allows for the derivation of a cause of death dataset from an input clinical dataset. Clinical data is broadly available in the United States in deidentified form and can be sourced from medical and pharmacy insurance claims, electronic health records, laboratory information systems, and other data capture systems used in clinical and medical payment operations. Further, this data can be aggregated to form a clinical dataset that is longitudinal across a patient's medical history, as well as broad in coverage with deidentified records for millions of patients.

In accordance with embodiments of the present disclosure, a method for deriving primary and secondary causes of death from clinical data includes the application of a series of processes upon an input clinical data set. These processes can include, for example, the identification of individuals (where the personal information of said individuals are in identified or deidentified form) within the clinical data set that are deceased, the mapping and aggregation of clinical codes within the deceased individuals' clinical history to a standardized taxonomy of clinical codes that are related to mortality and specific causes of death, the application of acuity modifiers to each clinical event and/or episode in the deceased individual's history to increase or decrease its inferred importance on the eventual cause of death, and the determination of primary and secondary causes of death based on the aggregate weighting of clinical event(s) and its acuity modifier(s).

In some implementations, the methods described herein are applied to clinical data where that data is stored (locally or in the cloud); in other implementations, the methods described herein are applied to clinical data that is sent to it as a single record or batch file, and a cause of death is returned to the sender. Such a form of implementation may be executed through an application programming interface (API) or the exchange of input and output files, for example. In some implementations, the processes of the methods described herein are applied separately, and potentially by different parties or systems. In other implementations, the processes are applied within a single system. In some implementations, the processes are directed by a set of algorithmic rules. In still other implementations, the processes are directed by an algorithm that may be derived from machine learning, artificial intelligence, or similar methodologies, for example.

Embodiments of the present disclosure also allow for the derivation of confidence scores and/or confidence levels for each output cause of death as well as the measurement of quality, accuracy, completeness, and other features of the data (clinical, reference and mapped) at each step of the process. These measurements may be used singly or in combination to assess the veracity of each step in the process, and to generate an overall confidence score or level that can be reported with the output cause of death.

### BRIEF DESCRIPTION OF THE DRAWINGS

FIG. 1 is a schematic diagram in accordance with embodiments of the present disclosure.

FIG. 2 is a flow diagram illustrating a cause of death determination process according to embodiments of the present disclosure.

### DETAILED DESCRIPTION OF EMBODIMENTS

The presently disclosed subject matter now will be described more fully hereinafter with reference to the accompanying drawings, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated drawings. Therefore, it is to be understood that the presently disclosed subject matter is not to be limited to the specific embodiments disclosed and that modifications and other embodiments are intended to be included within the scope of the appended claims.

It will be appreciated that reference to “a”, “an” or other indefinite article in the present disclosure encompasses one or a plurality of the described element. Thus, for example, reference to a weight may encompass one or more weights, reference to an individual may encompass one or more individuals and so forth.

For purposes of the present disclosure, the term “likelihood” can be considered to mean a quantitative measure that represents the probability that a specific disease indication is the cause of death of an individual, considering the significance, severity and associated treatment provided to the individual during healthcare events in the individual's life. The calculated likelihood can be based on clinical factors, their acuity weights, the frequency of occurrences and their proximity to the date of death, for example.

For purposes of the present disclosure, the term “confidence score” can be used interchangeably with the term “confidence level” or “level of confidence” and can be considered to mean a quantitative and/or categorical measure that quantifies the reliability and comprehensiveness of, for example, the underlying real-world data (RWD) to determine the specific disease indication as cause of death. The confidence score can be calculated from the completeness, quality and accuracy/validity of the RWD available to indicate the strength of evidence supporting the specific disease indications and determined likelihoods as described herein for each cause of death, for example.

For purposes of the present disclosure, the term “acuity weights” can be considered to mean numerical values assigned to clinical factors based on the severity and criticality of medical events or episodes. These weights quantify the impact of each factor on the likelihood of a disease indication being the cause of death, reflecting the relative importance of clinical events, considering their intensity, urgency, and associated risk of mortality. They can be calculated using a heuristic model, which assesses the frequencies of clinical factors in deceased versus living populations to determine their relative significance of each in contributing to the likelihood of death. These acuity weights can be recalculated using additional data or tailored based on desired use-cases or patient populations.

For purposes of the present disclosure, the term “clinical factors” can be considered to mean variables or characteristics related to a patient's medical history, health status, and treatment. These factors include treatment provider, location of treatment, symptoms, diagnostic test results, medical procedures, comorbidities, and treatment responses, for example.

Embodiments of the present disclosure can be employed in epidemiology and medical research. For example, the inclusion of cause of death within RWD can support identification and prioritize leading clinical causes of death in different populations to assist researchers in understanding the final outcomes of disease patterns and any specific interventions applied.

Embodiments of the present disclosure can further be employed in clinical decision support. Healthcare providers can use the cause of death as an analysis endpoint for gaining insights on diagnostic accuracy, treatment support and patient management.

Embodiments of the present disclosure can further be employed in insurance and actuarial analysis. Knowledge of the most likely cause of death can support insurance companies and actuaries in their risk assessment and underwriting process to determine rates, evaluate policy risks and predict the impact from claims.

Embodiments of the present disclosure can further be employed in public health. Public health agencies can utilize the cause of death to understand, monitor and report on causes of death at the population level directly from claims data instead of relying on death certificates. This can be used to inform policy, resource allocation and target health interventions.

Although the present disclosure will be described with reference to the example embodiment or embodiments illustrated in the drawings, it should be understood that many alternative forms can embody the present disclosure. One of skill in the art will additionally appreciate different ways to alter the parameters of the embodiment(s) disclosed, in a manner still in keeping with the spirit and scope of the present disclosure.

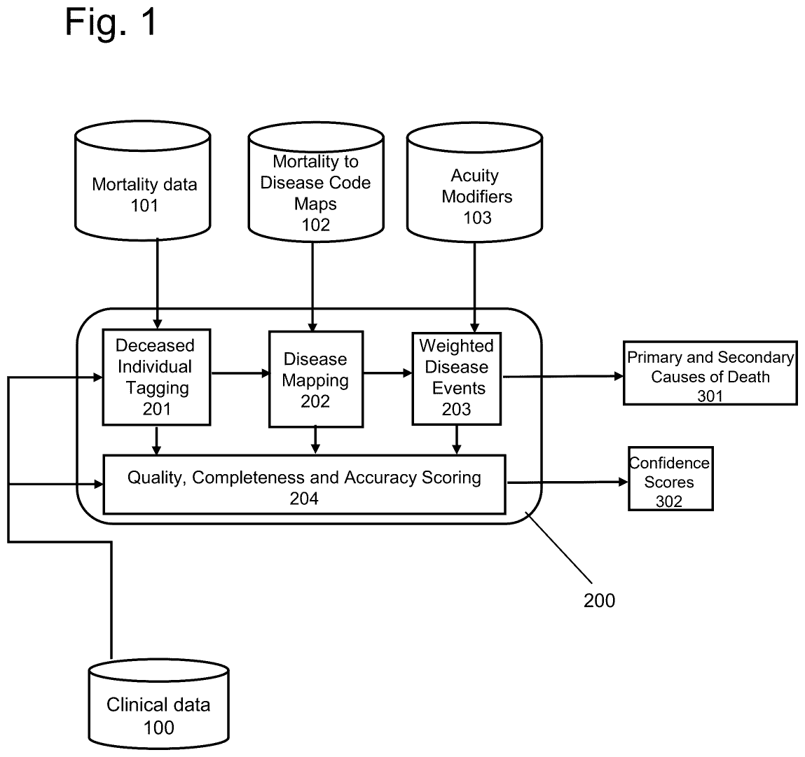

FIG. 1 depicts clinical data 100 as an input to the cause of death system 200 according to embodiments of the present disclosure. Clinical data 100 may encompass clinical, procedural, and/or diagnostic histories, sourced from multiple data collection points such as Electronic Health Records (EHRs), pharmacies, laboratories, or insurance claims, for example, also known as real-world data (RWD). Clinical data may contain information about clinical care provided (e.g., codes for procedures and diagnoses), service provider specialties, clinical care code modifiers, places of clinical service, and billing information associated with a person. As one skilled in the art will appreciate, clinical data 100 may be read into the cause of death system 200 through multiple means, including ingestion in batch into a database, of a single record through an API call, or other processes.

When clinical data 100 contains information for more than just deceased persons, embodiments of the cause of death system 200 according to the present disclosure can segregate, mark, or otherwise tag the data pertaining to deceased individuals from that of individuals who are alive through a deceased individual tagging process 201. This deceased individual tagging process 201 uses as input the clinical data 100 and a reference set of mortality data 101, for example. Mortality data 101 can include personally-identifying information (PII) such as the name, date of birth, address, phone number, or other data that can be used to discretely represent a specific deceased individual, as well as information about the mortality event such as the date of death, location of death, and similar. Mortality data 101 can be derived from public sources such as obituaries, private sources such as commercial databases, or government sources such as state vital records or death certificates. The deceased individual tagging process 201 matches the individuals in the mortality data 101 to the individuals in the clinical data 100. In various embodiments, wherever a record is found for an individual in the clinical data 100 and in the mortality data 101, the deceased individual tagging process 201 will add an indicator to that record that the individual is deceased. A record from clinical data 100 can be matched to the records in mortality data 101 according to various approaches, including incorporating different algorithms using different specific elements of the PII with different weights to determine what is counted as a match, as well as methods in which the records in both mortality data 101 and clinical data 100 are deidentified or processed to remove, mask, or obscure the PII in each and the identifying information is replaced with a unique identification number, token, or similar, upon which the matching process is run.

For each individual marked as deceased by the deceased individual tagging process 201, the cause of death system 200 retrospectively examines the clinical data 100 from the date of death determined by the mortality data 101 to catalog the clinical events that occurred leading up to the mortality event. These clinical events can be standardized and aggregated by a disease mapping process 202, for example, which uses as input mortality to disease code maps reference data set 102. Effective disease mapping ensures accurate data and event aggregation and reporting across multiple sources and coding systems to ensure that different codes and coding systems referring to the same clinical condition are appropriately mapped. Mortality to disease code maps 102 may consist of any number of medical coding systems available in the industry, or as a customized taxonomy of medical concepts. As one example implementation, the mortality to disease code map 102 can include the mapping of international classification of diseases (ICD), current procedural terminology (CPT), and other healthcare codes to the published guidelines of United States Centers for Disease Control & Prevention (CDC) recognized cause(s) of death. In another implementation, the mortality to disease code maps 102 could include mapping ICD, CPT, healthcare common procedure coding system (HCPCS), and logical observation identifiers names and codes (LOINC) to unified medical language system (UMLS) concepts. In another implementation, the mortality to disease code maps 102 could involve mapping ICD, CPT, HCPCS, SNOMED CT (Systematized Nomenclature of Medicine-Clinical Terms), RxNorm (for standardizing drug names), LOINC, and other coding systems to the standardized vocabularies used in the OMOP (Observational Medical Outcomes Partnership) Common Data Model, ensuring comprehensive integration of diagnoses, procedures, medications, and laboratory measurements In still other implementations, the user may be given the ability to integrate their own disease concepts (e.g., ICD codes) into bespoke mortality to disease code maps 102; for example, the user may replace the default mapping of ICD code E10.9 (Type 1 diabetes mellitus without complications) to the CDC-defined cause-of-death category of “Diabetes mellitus” with a new mapping of E10.9 and a number of other related disease codes to a new mortality group called “Metabolic disease”. In other implementations, the mortality to disease code maps 102 allow for the aggregation of different code systems to varying degrees of granularity, where a hierarchical code system is employed; the CDC-defined cause of death category, the CDC-defined113 ICD-10 category, the ICD chapter, or ICD sub chapter, for example.

In various embodiments, the disease mapping process 202 reads the clinical code(s) (ICD, CPT, etc.) in the deceased individual's clinical data 100 and maps it to the standardized concept in the mortality to disease code map 102. As one skilled in the art will appreciate, this mapping process can be applied in many ways. In some example implementations, this disease mapping process 202 may replace the value in the clinical data 100 with the newly mapped value, or the disease mapping process 202 may create a new record for the deceased individual where the newly mapped values from the mortality to disease code map 102 are appended along with other relevant information from the originating clinical data 100 such as service dates, treating physician, or facility, as well as information such as date of death from mortality data 101.

The cause of death system 200 uses the mapped clinical data for deceased individuals (i.e., the output of the deceased individual tagging process 201 and disease mapping process 202) to determine the likely cause(s) of death 301. Not each clinical event in the deceased individual's history is necessarily equally relevant to determining the cause of death. In various embodiments of the present disclosure, each clinical event can be assigned a weight by the cause of death system 200 using a set of rules and calculated information contained in a reference data set of acuity modifiers 103. The acuity modifiers 103 are factors or rules or calculated values that represent the severity and urgency associated with certain diseases, conditions, procedures and associated treatment, where treatment includes the healthcare provider and setting. It will be appreciated that the acuity modifiers can be rules, factors, mathematical computations, scores and/or other values. According to various embodiments of the present disclosure, the specific factors or rules used to assign weights to clinical events can vary dramatically and can in some implementations be applied as a set of fixed expertly determined rules, calculated deterministically or as a dynamic model such as those borne of artificial intelligence or machine learning systems, or other techniques or inputs. In an example implementation, acuity modifiers 103 are assigned based on the relative recency of a clinical event to the time of death and are assigned different weights based on whether the indication is chronic or acute, or based on whether diagnosis occurred during a hospital admission. In another example implementation, the acuity modifiers 103 may establish the severity of a specific disease found in the clinical data 100 and may take into consideration other factors found in clinical data 100 such as the timing of the diagnosis, the location of service where the diagnosis was made, and the specialty or experience of the diagnosing provider. Moreover, procedures occurring during the episode of care and any associated modifiers may be also taken into consideration by the acuity modifiers 103 or assigned distinct specific acuity weightings independently of other events during the episode of care. In another example, billing service provider specialty, rendering provider, referring provider, and facility type may be used to assign an acuity modifier 103 to a clinical event. For example, a record of care in clinical data 100 that includes a highly trained clinical specialist (e.g., cardiologists and oncologists) may receive a higher acuity score in the acuity modifier 103 module. In yet another example, specialized and acute places of service (e.g., emergency room (ER) or inpatient hospital) may be assigned a higher acuity score than primary care locations (e.g., a clinic and doctor's office), or a procedure modifier that indicates anesthesia and surgery was required as part of treatment may also be assigned a higher acuity score. In still other implementations, numerous acuity modifiers 103 may be combined to create an overall acuity modifier for an event or episode of care in the clinical data 100. In addition, the acuity modifier 103 module may allow a user to add to, or modify, the specific acuity modifiers they wish to apply based on their unique patient populations or research interests or expert knowledge of the clinical event(s).

For any or all individuals, the cause of death system 200 may apply the acuity modifiers 103 to the mapped clinical data 100 for deceased individuals to create a resulting weighted disease events 203 output. Through this process, each relevant clinical event is assigned a higher or lower weight which quantifies the likelihood that the disease indication is a cause of death for that individual. The cause of death system 200 can integrate events across multiple years of clinical data 100 to determine and rank probable causes of death, determined by the likelihood metric, in an output dataset or report of primary and, optionally secondary, causes of death 301.

The process of integrating weighted disease events 203 according to the present disclosure can be informed and implemented in numerous ways. In one example implementation, the cause of death system 200 can rank the weighted disease events 203 from highest to lowest and report the top event (most likely) as the primary cause of death, and the lesser weighted event(s) as one or more secondary causes of death. In another implementation, the cause of death system 200 can select only weighted disease events 203 which have a weight above a system-or user-defined threshold to report in the primary and secondary causes of death 301 output. In still other implementations, the cause of death system 200 may compare the weighted disease events 203 with an external reference file and report to the primary and secondary causes of death output 301 only those events that have weights that are above a specific threshold and also match attributes in the external reference file. In still other implementations, the cause of death system 200 may allow a user to adjust the process for integrating the weighted disease events 203 to suit the needs and focus of their respective studies. In various embodiments, a report or output can be, or can be included in, a notification over a network, such as an electronic communication to a communications device, such as a mobile communications device or other computing device. In various embodiments, the notification can be issued when a threshold confidence level is met or exceeded for a given determination, such as a likelihood that one or more clinical factors comprising clinical coding in medical data that occurred leading up to a mortality event for an individual is a primary cause of death for the individual, for example.

According to various embodiments, determining a likelihood that one or more clinical factors is a cause of death includes applying one or more acuity weights to each of the one or more clinical factors. In various embodiments, determining a likelihood that one or more clinical factors is a primary cause of death is based on aggregate weighting of each clinical factor and one or more acuity weights applied to each clinical factor. It will be appreciated that in addition to applying one or more acuity weights based on the relative recency of one or more clinical factors to a time of death for an individual, one or more acuity weights can be applied based on the duration of the disease, the severity of a specific disease found in the dataset, a timing of a diagnosis, a location of service where the diagnosis was made, whether the one or more clinical factors is chronic or acute, an identity of an associated provider, one or more procedures occurring during an episode of care, a billing service provider specialty, a rendering provider, a referring provider, and a facility type. According to various embodiments of the present disclosure, multiple acuity weights can be combined to create an overall acuity weight for one or more clinical events. Further the acuity weights applied to each clinical factor can be based on the individual being associated with a unique patient population or a unique clinical event.

During any or all of the processes conducted by the cause of death system 200, additional scores or metrics can be derived that may be used as quality, completeness, and accuracy scores 204. The implementation and collection of these scores can take a variety of forms. In one example implementation, the clinical data 100 can be scored based on data recency at an individual and aggregate level, the completeness of information (i.e., fill rates of clinical care information), and the accuracy of information provided (e.g., whether the codes in the clinical data 100 are valid codes). In another example implementation, the accuracy of the deceased individual tagging 201 process may be scored based on the type of matching that was used, and how many of the identifying or token values matched between the mortality data 101 and the clinical data 100 to determine that the records belonged to the same individual. The score or scores from each process may be assembled or associated with different values in the cause of death solution 200; for example, scores may be associated with specific deceased individuals 201, mapped diseases 202, weighted clinical events 203, or similar. In another example implementation, the quality of the input clinical data 100 to the cause of death solution 200 is measured based on the diversity, specificity and volume of the data values contained within.

Using the quality, completeness, and accuracy scoring 204 values, embodiments of the cause of death system 200 can generate confidence scores 302 to help end-users determine their own reliance on the algorithm's results. It will be appreciated that confidence scores 302 may be implemented in different ways. In one example implementation, a confidence score may be assigned to a particular primary and secondary cause of death 301 given the available weighted disease events 203 combined with the quality, completeness, and accuracy scores 204 of the processes from which those events were derived. In another example implementation, the confidence score 302 may include a RWD fitness score that reflects how well the available clinical data 100 can predict a cause of death, which is determined by comparing the historical accuracy of the primary and secondary cause of death 301 predictions derived from this type of clinical data 100 at an aggregate level against the published CDC reports. In another example implementation, the confidence score 302 may include a RWD density Score, a metric that evaluates the depth and breadth of available clinical data 100 by assessing the number of non-null data points, the range or diversity of data across relevant clinical categories, and the temporal coverage of the data. In another example implementation, the confidence score 302 may include a patient coverage score, which assesses the availability of clinical data 100 per patient across health domains over time, ensuring that predictions are made based on a holistic view of the patient's health. In still another example implementation, the confidence score 302 may include a time-relevance score that evaluates the accuracy of the primary and secondary causes of death 301 prediction in relation to the temporal proximity of the weighted clinical event 203 used for prediction to the actual time of death reported in mortality data 201.

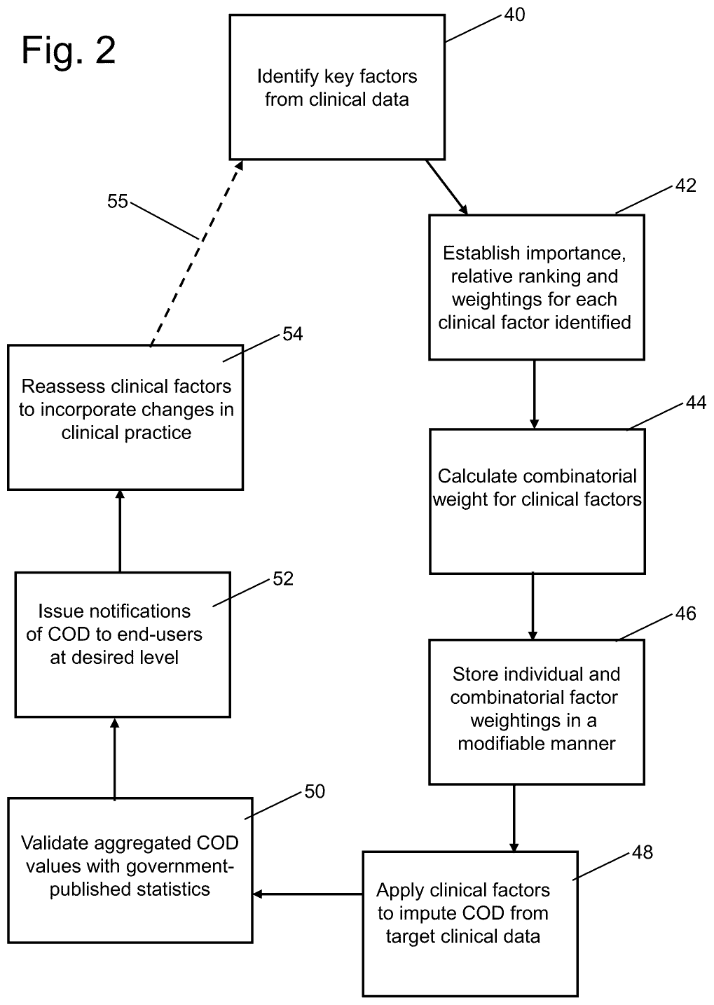

FIG. 2 is a flow diagram illustrating a cause of death process according to embodiments of the present disclosure. As shown in FIG. 2, at 40, key factors from clinical data are identified. Examples of such clinical data are described above with regard to clinical data 100. As at 42, for each clinical factor identified, importance, relative ranking and weightings are established. As at 44, a combinatorial weight for the clinical factors is calculated. As at 46, individual and combinatorial factor weightings are stored in a modifiable manner. As at 48, clinical factors are applied to impute cause of death from target clinical data. As at 50, aggregated cause of death values are validated with government-published statistics. As at 52, notifications of cause of death can be sent to end-users at a desired level such as described elsewhere herein. As at 54, clinical factors to incorporate changes in clinical practice can be reassessed. In optional embodiments, as depicted in dashed line 55, after the reassessment in 54, the key factors from clinical data can be re-performed as at 40 and the process repeated.

In certain embodiments, a system according to the present disclosure includes a computing device (such as a server) that includes at least one processor and at least one memory device or data storage device. As further described herein, the computing device includes at least one processor configured to transmit and receive data or signals representing events, messages, commands, or any other suitable information between the computing device and other devices. The processor of the computing device is configured to execute the events, messages, or commands represented by such data or signals in conjunction with the operation of the computing device. In various embodiments, the computing device incorporates, or interacts with outside systems having, software for: predicting actuarial risk, improved accuracy of drug effectiveness determinations, improved accuracy of survival curves, and other applications.

As will be appreciated by one skilled in the art, aspects of the present disclosure may be illustrated and described herein in any of a number of patentable classes or context including any new and useful process, machine, manufacture, or composition of matter, or any new and useful improvement thereof. Accordingly, aspects of the present disclosure may be implemented entirely hardware, entirely software (including firmware, resident software, micro-code, etc.) or combining software and hardware implementation that may all generally be referred to herein as a “circuit,” “module,” “component,” or “system.” Furthermore, aspects of the present disclosure may take the form of a computer program product embodied in one or more computer readable media having computer readable program code embodied thereon. Embodiments of the system and method described herein are specifically configured to provide a technical solution to a particular problem utilizing an unconventional combination of steps/operations to carry out aspects of the present disclosure. In particular, the system and method implement a unique combination of steps to provide a novel approach to determining cause of death in individuals (or records for individuals) employing deidentified information for those individuals.

It will be appreciated that any combination of one or more computer readable media may be utilized in accordance with the present disclosure. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing, including a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

It will be appreciated that all of the disclosed methods and procedures herein can be implemented using one or more computer programs or components. These components may be provided as a series of computer instructions on any conventional computer-readable medium, including RAM, SATA DOM, or other storage media. The instructions may be configured to be executed by one or more processors which, when executing the series of computer instructions, performs or facilitates the performance of all or part of the disclosed methods and procedures.

Unless otherwise stated, devices or components of the present disclosure that are in communication with each other do not need to be in continuous communication with each other. Further, devices or components in communication with other devices or components can communicate directly or indirectly through one or more intermediate devices, components or other intermediaries. Further, descriptions of embodiments of the present disclosure herein wherein several devices and/or components are described as being in communication with one another does not imply that all such components are required, or that each of the disclosed components must communicate with every other component. In addition, while algorithms, process steps and/or method steps may be described in a sequential order, such approaches can be configured to work in different orders. In other words, any ordering of steps described herein does not, standing alone, dictate that the steps be performed in that order. The steps associated with methods and/or processes as described herein can be performed in any order practical. Additionally, some steps can be performed simultaneously or substantially simultaneously despite being described or implied as occurring non-simultaneously.

It will be appreciated that algorithms, method steps and process steps described herein can be implemented by appropriately programmed computers and computing devices, for example. In this regard, a processor (e.g., a microprocessor or controller device) receives instructions from a memory or like storage device that contains and/or stores the instructions, and the processor executes those instructions, thereby performing a process defined by those instructions. Furthermore, aspects of the present disclosure may take the form of a computer program product embodied in one or more computer readable media having computer readable program code embodied thereon.

Computer program code for carrying out operations for aspects of the present disclosure may be written in any combination of one or more programming languages, including an object oriented programming language such as Java, Scala, Smalltalk, Eiffel, JADE, Emerald, C++, C #, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on a user's computer, partly on a user's computer, as a stand-alone software package, partly on a user's computer and partly on a remote computer or entirely on the remote computer or server.

Where databases are described in the present disclosure, it will be appreciated that alternative database structures to those described, as well as other memory structures besides databases may be readily employed. The drawing figure representations and accompanying descriptions of any exemplary databases presented herein are illustrative and not restrictive arrangements for stored representations of data. Further, any exemplary entries of tables and parameter data represent example information only, and, despite any depiction of the databases as tables, other formats (including relational databases, object-based models and/or distributed databases) can be used to store, process and otherwise manipulate the data types described herein. Electronic storage can be local or remote storage, as will be understood to those skilled in the art. Appropriate encryption and other security methodologies can also be employed by the system of the present disclosure, as will be understood to one of ordinary skill in the art.

Although the present approach has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present approach.

Among other aspects described herein and as shown in the drawings, embodiments of the present disclosure provide a system and method for determining one or more causes of death from clinical data. The clinical data is deidentified with one or more unique identifiers for each record, and the derived cause of death is appended, directly or indirectly, to those identifiers. In various embodiments, a confidence score or confidence level is determined for a derived cause of death and/or for the aggregate cause of death output. In various embodiments, the system employs an API and the method is applied through an API call. According to various embodiments, aspects of the method are applied in separate processes. Further, aspects of the method as described herein need not be performed while still achieving the desired outcome and/or result. It will be appreciated that portions of the process are informed by user-input data or business rules.

Embodiments of the present disclosure may be embodied in other specific forms without departing from the spirit or essential characteristics thereof. The present embodiments are therefore to be considered in all respects as illustrative and not restrictive, the scope of the invention being indicated by the claims of the application rather than by the foregoing description, and all changes which come within the meaning and range of equivalency of the claims are therefore intended to be embraced therein.

1. A system for determining one or more causes of death for an individual, comprising:
a processor;
a memory storing instructions that, when executed by the processor, cause the processor to:
tag one or more records from a dataset to denote that an individual associated with the one or more records is deceased;
for one or more tagged records, determine a likelihood that one or more clinical factors comprising clinical coding in medical data that occurred leading up to a mortality event for the individual associated with the one or more records is a primary cause of death for the individual; and
upon a confidence level for the determined likelihood meeting a threshold level, issue a notification.

2. The system of claim 1, wherein the clinical factors further comprise data regarding at least one of: a setting of a medical event associated with the clinical coding, a treatment received, a treatment provider, a location of treatment, a symptom, a diagnostic test result, a comorbidity and a treatment response.

3. The system of claim 1, wherein tagging one or more records comprises matching one or more records in a mortality dataset to the one or more records from the dataset.

4. The system of claim 1, wherein determining a likelihood that one or more clinical factors is a cause of death comprises mapping and aggregating clinical codes within the one or more clinical factors to a standardized taxonomy of clinical codes that are related to mortality and specific causes of death.

5. The system of claim 1, wherein determining a likelihood that one or more clinical factors is a cause of death comprises applying one or more acuity weights to each of the one or more clinical factors.

6. The system of claim 5, wherein determining a likelihood that one or more clinical factors is a primary cause of death is based on aggregate weighting of each of the one or more clinical factors and the one or more acuity weights applied to each of the one or more clinical factors.

7. The system of claim 5, wherein the dataset comprises a clinical dataset and wherein mapping the clinical codes comprises replacing a value in the clinical dataset with a newly mapped value.

8. The system of claim 5, wherein the dataset comprises a clinical dataset and wherein mapping the clinical codes comprises creating a new record for the deceased individual where newly mapped values are appended from the clinical dataset.

9. The system of claim 5, wherein the one or more acuity weights is applied based on the relative recency of the one or more clinical factors to a time of death for the individual.

10. The system of claim 5, wherein the one or more acuity weights is applied based on the duration of the disease.

11. The system of claim 5, wherein the one or more acuity weights is applied based on a severity of a specific disease found in the dataset.

12. The system of claim 5, wherein the one or more acuity weights is applied based on at least one of: a timing of a diagnosis, a location of service where the diagnosis was made, whether the one or more clinical factors is chronic or acute, and an identity of an associated provider.

13. The system of claim 5, wherein the one or more acuity weights is applied based on one or more procedures occurring during an episode of care.

14. The system of claim 5, wherein the one or more acuity weights is applied based on at least one of: a billing service provider specialty, a rendering provider, a referring provider, and a facility type.

15. The system of claim 5, wherein a plurality of the one or more acuity weights is combined to create an overall acuity weight for the one or more clinical events.

16. The system of claim 5, further comprising adapting the one or more acuity weights applied to each of the one or more clinical factors based on the individual being associated with a unique patient population or a unique clinical event.

17. The system of claim 5, wherein the acuity weights are derived from clinical data or an individual being associated with a unique patient population or a unique clinical event.

18. The system of claim 1, further comprising determining a veracity level comprising the quality, accuracy and completeness of the dataset.

19. The system of claim 1, further comprising determining one or more secondary potential causes of death for the individual.

20. The system of claim 1, further comprising determining a level of confidence for the primary cause of death and the secondary potential causes of death.

21. The system of claim 20, wherein the level of confidence comprises a veracity level comprising the quality, accuracy and completeness of the dataset.

22. The system of claim 20, wherein the level of confidence comprises a score comprising a real-world fitness score or a real-world density score.

23. The system of claim 20, wherein the level of confidence comprises a time-relevance score that evaluates the accuracy of the causes of death prediction in relation to a temporal proximity of one or more of the clinical factors to an actual time of death of the individual.

24. A computer-implemented method for determining one or more causes of death for an individual, comprising:
tagging, by a cause of death component, one or more records from a dataset to denote that an individual associated with the one or more records is deceased;
for one or more tagged records, determining, by the cause of death component, a likelihood that one or more clinical factors comprising clinical coding in medical data that occurred leading up to a mortality event for the individual associated with the one or more records is a primary cause of death for the individual; and
upon a confidence level for the determined likelihood meeting a threshold level, issuing a notification.
